(19) United States
(12) Patent Application Publication
(10) Pub. No.: US 2023/0405190 A1
(43) Pub. Date: Dec. 21, 2023

(54) Composite Materials for Uses in Cardiac and Other Tissue Repair

(71) Applicant: Emory University, Atlanta, GA (US)

(72) Inventors: Sai Muralidhar Padala, Atlanta, GA (US); Jahnavi Mudigonda, Roswell, GA (US)

(21) Appl. No.: 18/035,484

(22) PCT Filed: Nov. 4, 2021
(86) PCT No.: PCT/US2021/058063

§ 371 Date: May 4, 2023

Related U.S. Application Data
(60) Provisional application No. 63/109,676, filed on Nov. 4, 2020.

(51) Int. Cl. A61L 27/54; D01F 6/92; D01D 5/00; D01F 1/10; A61L 27/36; A61K 31/722; A61K 31/365

(52) U.S. Cl. CPC A61L 27/54; D01F 6/92; D01D 5/0084; D01F 1/10; A61L 27/3625; A61K 31/722; A61L 2300/216; D10B 2331/041; D10B 2509/00; D10B 2401/12; A61L 2430/20; A61L 2300/232; A61K 31/365

(57) ABSTRACT

This disclosure relates to composite materials for repairing a tissue of a subject by implanting the composite material in or on the tissue, e.g., heart tissue. In certain embodiments, the composite material comprises a first material and a second material coated on the first material, wherein the first material is an inert substantially non-biodegradable material providing mechanical support; wherein the second material is a biodegradable active material attracting cells from the body of a subject, and wherein, when the composite material is implanted, cells integrate into the inert material providing a remodeled tissue. In certain embodiments, this disclosure relates to methods of using the composite material wherein the inert and active materials allow for remodeling of the material in vivo.

### BACKGROUND

Tetralogy of Fallot is diagnosed when a newborn is identified as having a combination of heart defects (congenital) usually resulting blue-tinged skin associated with a lack of oxygen in the blood. When open-heart surgery is needed to correct this condition, it typically involves closing a ventricular septal defect with a patch and placing a patch on the right ventricle and main pulmonary artery to improve blood flow to the lungs. Surgery is often curative, but not universally effective. Complications include blood clots (which may be in the brain causing stroke), infection in the lining of the heart and heart valves (bacterial endocarditis), abnormal heart rhythms (arrhythmias), and heart failure. Thus, there is a need to identify improved methods and materials.

Materials, which are inert at the time of implantation, but over time hone the cells of the host, remodel into the tissue of patients, and are fully replaced, would be optimal in cardiovascular surgery. However, most synthetic materials lack durability due to structural deterioration or thrombose over time.

CorMatrix® is a company that advertises extracellular matrix (ECM) products made from porcine small intestinal submucosa used as an acellular biologic scaffold in surgical applications. See Product Brochure, CorMatrix® ECM™ Technology, Rethink the treatment of a damaged heart, (c) 2010, and U.S. Pat. Nos. 9,034,367 and 8,778,012.

Jahnavi et al. report a polymer layered bio-hybrid heart valve scaffold. Materials Science and Engineering C 51 (2015) 263-273.

Jahnavi et al. report biological and mechanical evaluation of a bio-hybrid scaffold for autologous valve tissue engineering. Materials Science and Engineering C 73 (2017) 59-71.

Cassan et al. report blending chitosan-g-poly(caprolactone) with poly(caprolactone) by electrospinning to produce functional fiber mats for tissue engineering applications. J. Appl. Polym. Sci. 2020, 137, 48650.

See also U.S. Published Patent App. Nos. 2014/0188218, 2015/0282811, US2018/0185144, and WTO PCT App. Nos. WO2010023463, WO2013096448, and WO2020214863.

References cited herein are not an admission of prior art.

### SUMMARY

This disclosure relates to composite materials for repairing a tissue of a subject by implanting the composite material in or on the tissue, e.g., heart tissue. In certain embodiments, the composite material comprises a first material and a second material coated on the first material, wherein the first material is an inert substantially non-biodegradable material providing mechanical support; wherein the second material is a biodegradable active material attracting cells from the body of a subject, and wherein, when the composite material is implanted, cells integrate into the inert material providing a remodeled tissue. In certain embodiments, this disclosure relates to methods of using the composite material wherein the inert and active materials allow for remodeling of the material in vivo.

In certain embodiments, the inert material is a synthetic or natural polymer. In certain embodiments, the inert material is derived from tissues of an animal or human.

In certain embodiments, the active material is a synthetic or natural polymer. In certain embodiments, the active material attracts cells. In certain embodiments, the active material elutes a drug. In certain embodiments, the active material is hemocompatible. In certain embodiments, the active material is tunable to suit the environment once implanted into a subject.

In certain embodiments, the composite material is made by the process of dipping inert material into the active material. In certain embodiments, the composite material is made by the process of overlaying the active material on the inert material. In certain embodiments, the composite material is made by the process of weaving strands of the inert and active material with one another. In certain embodiments, the composite material is made by the process of weaving strands of the inert material over active material.

In certain embodiments, the inert material is decellularized pericardium from an animal or a human. In certain embodiments, the active material is a polymer fiber of caprolactone and chitosan. In certain embodiments, the polymer fiber of caprolactone and chitosan is about a 10:1 by weight ratio respectively. In certain embodiments, the active material has an average thickness of between 70 to 100 μm.

In certain embodiments, the composite material is made by the process of contacting the decellularized pericardium with a dialdehyde providing aldehyde functionalized decellularized pericardium, and contacting the aldehyde functionalized decellularized pericardium with a polymer, e.g., a polymer fiber of caprolactone and chitosan.

In certain embodiments, the properties of the composite material are not an additive result and are more than an additive result of the material properties of the individual inert and active materials. In certain embodiments, the properties of the inert or active material dominate the material properties of the composite. In certain embodiments, the properties of the inert or active material improve the properties of the composite material when combined in the composite.

In certain embodiments, this disclosure relates to methods of repairing a tissue comprising implanting an effective amount of a composite material as disclosed herein into a subject at a location of desired tissue growth in a subject in need thereof.

In certain embodiments, the location of desired tissue growth is in or on the heart of a subject diagnosed with a heart defect or developmental void. In certain embodiments, the subject is diagnosed with a congenital heart disease.

### BRIEF DESCRIPTION OF THE SEVERAL VIEWS OF THE DRAWINGS

FIG. 1A shows characterization of a Bio-Hybrid composite, polycaprolactone:chitosan blend, untreated bovine pericardium (BP), and decellularized BP by x-ray photon spectroscopy. Differences in binding energy correspond to peaks C═C, C═O, C—O and C—N.

FIG. 1B shows Fourier transform infrared spectroscopy of the Bio-Hybrid, polycaprolactone:chitosan blend, and decellularized BP showing unique peaks in the Bio-Hybrid corresponding to changes in C═C, C═O, N—H and C—H groups.

FIG. 2 shows data on peel profiles of the Bio-Hybrid composite showing the load measured to peel the polymer in the Bio-Hybrid from the decellularized pericardium. The polymer was peeled using a clip attached to a weighted hanger and a pulley. The Bio-Hybrid composite conduit was subjected to low shear and high shear (a portion of sample constricted to about 50% of original diameter) to emulate what is experienced by a normal artery (15 dynes/cm 2) in a continuous flow loop.

FIG. 3 shows data of in-vitro mechanical properties of the untreated BP, decellularized pericardium, and the Bio-Hybrid composite in terms of uniaxial tensile strength (kPa) of the materials indicating no difference in the tensile strength and a significant increase in the extensibility in the Bio-Hybrid.

FIG. 3B shows data on the uniaxial tensile extensibility ratio.

FIG. 3C shows data from equibiaxial (10%) testing of the three groups showing a stiffer response of the Bio-Hybrid in the circumferential direction and similar response of the fresh and decellularized pericardium.

FIG. 3D shows data in the longitudinal direction.

FIG. 3E shows data from step biaxial testing with 10% strain in the circumferential direction of testing indicating a stiffer response of Bio-Hybrid than other two group whereas the decellularized is more compliant in the circumferential direction.

FIG. 3F shows data in the longitudinal direction.

FIG. 3G shows data from step biaxial testing with 20% strain in the direction of testing indicating a stiffer response of Bio-Hybrid in the circumferential direction followed by untreated and decellularized BP.

FIG. 3H shows data in the longitudinal direction indicating a stiffer response of decellularized with absence of aligned polymer nanofibers in the Bio-Hybrid.

### DETAILED DISCUSSION

Before the present disclosure is described in greater detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be limited only by the appended claims. Although the function of certain compositions disclosed herein are believed to operate by particular mechanisms, it is not intended that embodiments of this disclosure be limited by any specific mechanism.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure refers to an example and infers that the example is not necessarily limited to the example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to the idea of excluding certain prior art element(s) as an inventive feature of a claim, but which may contain additional composition components or method steps, etc., that do not materially affect the basic and novel characteristic(s) of the compositions or methods, compared to those of the corresponding compositions or methods disclosed herein.

“Subject” refers to any animal, preferably a human patient, livestock, rodent, monkey, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, the term “biodegradable” in reference to a material refers to a molecular arrangement in the material that when implanted to a subject, e.g., human, will be broken down by biological mechanism such that a decomposition of the molecular arrangement will occur and the molecular arrangement will not persist for over a long period of time, e.g., the molecular arrangement will be broken down by the body after a several hours or days. In certain embodiments, the disclosure contemplates that the biodegradable material will not exist after a week or a month.

As used herein, the terms “substantially non-biodegradable” in reference to a material refers to a molecular arrangement in the material that when implanted to a subject, e.g., human, will be not be readily broken down by biological mechanism and the molecular arrangement will persist for over a long period of time, e.g., the molecular arrangement will not be entirely broken down by the body after month. However, it is contemplated that such material may be reconstructed through cellular processes after extremely long periods of time, e.g., several months or a year.

The terms “pharmacological agent”, “pharmaceutical agent”, “agent”, “active agent”, “drug”, and “pharmaceutical composition” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans, and primates; domestic household or farm animals, such as cats, dogs, sheep, goats, cattle, horses, and pigs; laboratory animals, such as mice, rats and guinea pigs; fish; reptiles; zoo and wild animals; and the like. Examples include statins, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds that modulate cell migration, compounds that modulate proliferation and growth of tissue, and vasodilating agents.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e., the protective tissue response to injury or destruction of tissues, which serves to destroy, dilute, or wall off both the injurious agent and the injured tissues. Examples include alclofenac, alclometasone dipropionate, alpha amylase, amcinafal, amfenac sodium, anakinra, anirolac, balsalazide disodium, bendazac, benoxaprofen, bromelains, broperamole, budesonide, carprofen, cliprofen, clobetasol propionate, clobetasone butyrate, clopirac, cortodoxone, decanoate, deflazacort, depo-testosterone, desonide, desoximetasone, dexamethasone dipropionate, diclofenac potassium, diclofenac sodium, diflorasone diacetate, diflumidone sodium, diflunisal, difluprednate, dimethyl sulfoxide, enolicam sodium, etodolac, felbinac, fenamole, fenbufen, fenclofenac, fendosal, fenpipalone, fentiazac, flazalone, flufenamic acid, flunisolide acetate, flunixin, flunixin meglumine, fluorometholone acetate, flurbiprofen, fluticasone propionate, furaprofen, halcinonide, halobetasol propionate, ibuprofen, ibuprofen aluminum, ibuprofen piconol, indomethacin, indomethacin sodium, indoprofen, isoxepac, isoxicam, ketoprofen, lofemizole hydrochloride, loteprednol etabonate, meclofenamate sodium, meclofenamic acid, mefenamic acid, mesalamine, methenolone, methenolone acetate, nabumetone, nandrolone, naproxen, naproxen sodium, naproxol, olsalazine sodium, oxaprozin, oxyphenbutazone, oxymetholone, pirfenidone, piroxicam, piroxicam cinnamate, piroxicam olamine, pirprofen, proquazone, proxazole, proxazole citrate, salsalate, stanozolol, sudoxicam, sulindac, suprofen, talniflumate, tenidap, tenidap sodium, tenoxicam, testosterone, testosterone blends, tiopinac, tixocortol pivalate, tolmetin, tolmetin sodium, triclonide, triflumidate, zidometacin, and zomepirac sodium.

### Composite Materials

This disclosure relates to composite materials for implanting into or on tissue, fascia, heart, or circulatory system of a subject comprising a first material and a second material coated on the first material, wherein the first material is an inert substantially non-biodegradable material providing mechanical support; wherein the second material is a biodegradable active material attracting cells from the body of a subject; and wherein the inert material attracts cells from the active material providing cells which hone into the inert material providing a remodeled tissue.

In certain embodiments, the inert material is a synthetic or natural polymer. In certain embodiments, the inert material is derived from tissues of an animal or human. In certain embodiments, the inert material is decellularized pericardium from an animal or a human.

In certain embodiments the inert material is a decellularized extra-cellular matrix obtained from an allogeneic or xenogeneic tissue sources. In certain embodiments, decellularized extra-cellular matrix is derived from cardiac sources (such as myocardium and pericardium) or non-cardiac sources (small intestinal submucosa and urinary bladder matrix). In certain embodiments the inert material is a collagen-rich biological tissue optionally containing glycoproteins and/or glycosaminoglycans. In certain embodiments the inert material is human pericardium or decellularized human pericardium.

In certain embodiments, the inert material is autologous human pericardium or decellularized human pericardium, or glutaraldehyde-treated decellularized pericardium coated with the active material. In certain embodiments, the inert material is derived from neonatal decellularized pericardium. In certain embodiments, the inert material comprises agrin protein or exogenously added agrin protein.

In certain embodiments, the inert material is an extracellular matrix (ECM) material derived from mucosal layers and components, submucosal layers and components, muscularis layers and components, and/or basement membrane layers and components. It is contemplated that the ECM material is obtained from any mammalian tissue source, including, for example and without limitation, stomach tissue (e.g., stomach submucosa (SS)), small intestinal tissue (e.g., small intestinal submucosa (SIS)), large intestinal tissue, bladder tissue (e.g., urinary bladder submucosa (UBS)), liver tissue (e.g., liver basement membrane (LBM)), heart tissue (e.g., pericardium), lung tissue, kidney tissue, pancreatic tissue, prostate tissue, mesothelial tissue, fetal tissue, a placenta, a ureter, veins, arteries, heart valves with or without their attached vessels, tissue surrounding the roots of developing teeth, and tissue surrounding growing bone. It is further contemplated that the ECM can be obtained from one or more mammals including, for example and without limitation: humans, cows, pigs, dogs, sheep, cats, horses, rodents, and the like.

In certain embodiments, the inert material is decellularized pericardium made by the process of detergent extraction and/or enzymatic extraction with or without hypotonic and/or hypertonic washings, and/or physical treatments (agitation, sonication, mechanical pressure and/or freeze-thawing). In certain embodiments the inert material is decellularized extracellular matrix material wherein all or substantially all antigenic components (nucleic acids, cell membranes, cytoplasmic structures, lipids and/or soluble matrix) are removed providing the decellularized extracellular matrix material.

In certain embodiments the inert material is a synthetic collagen or collagen-glycosaminoglycan scaffold.

In certain embodiments the inert material has an average pore size diameter of 1000 to 5 μm, 1000 to 50 μm, 300 to 100 μm, or 200 to 50 μm, 100 to 2 μm, 20 to 2 μm, or 15 to 1 μm.

In certain embodiments, the active material is a synthetic or natural polymer. In certain embodiments, the active material attracts cells. In certain embodiments, the active material elutes a drug or growth factor. In certain embodiments, the drug is an anti-inflammatory agent or an anti-coagulant. In certain embodiments, the growth factor is vascular endothelial growth factor (VEGF), hepatocyte growth factor (HGF) and/or insulin-like growth factor-1 (IGF-1).

In certain embodiments, the active material is hemocompatible. In certain embodiments, the active material is tunable to suit the environment once implanted into a subject.

In certain embodiments, the composite material is made by the process of dipping inert material into the active material. In certain embodiments, the composite material is made by the process of overlaying the active material is on the inert material. In certain embodiments, the composite material is made by the process of weaving strands of the inert and active material with one another.

In certain embodiments, the active material is a copolymer fiber of caprolactone and chitosan. In certain embodiments, the copolymer fiber of caprolactone and chitosan fiber is about a 10:1 by weight ratio respectively. In certain embodiments, the polymer fiber of caprolactone and chitosan fiber is about a 100:1, 50:1, 20:1, 10:1, 5:1, or 2:1 by weight ratio weight or molecular ratio respectively.

In certain embodiments, the active material is a copolymer fiber of chitosan and an alternative to caprolactone such as polyethylenimine/chitosan (PEI/CS), poly [(R)-3-hydroxybutyric acid)]/chitosan (PHB/CS), or poly(Lactide-co-glycolide)/chitosan (PLGA/CS).

In certain embodiments, the active material is a copolymer fiber of caprolactone and an alternative to chitosan such as poly(l-lactic acid)/poly (ε-caprolactone) (PLLA/PCL), polyethyleneimine/poly (ε-caprolactone) (PEI/PCL), and poly (N-isopropylacrylamide)/poly (6-caprolactone) (PNIPAM/PCL).

In certain embodiments, the active material has an average thickness of between 70 to 100 μm. In certain embodiments, the active material has an average thickness of between 30 to 150 μm, 50 to 150 μm, 50 to 120 μm, 70 to 100 μm, or 80 to 90 μm.

In certain embodiments, the composite material is made by the process of contacting the decellularized inert material, e.g., decellularized pericardium, with a dialdehyde providing aldehyde functionalized decellularized material, and contacting the aldehyde functionalized decellularized material with a polymer, e.g., a polymer fiber of caprolactone and chitosan. In certain embodiments, the dialdehyde is an alkyl dialdehyde, glutaraldehyde (pentanedial), succinaldehyde (butanedial), or a dialdehyde starch.

In certain embodiments, the properties of the composite material function in a way that are not an additive result and are more than an additive result of the material properties of the individual inert and active materials. In certain embodiments, the properties of the inert or active material dominate the material properties of the composite. In certain embodiments, the properties of the inert or active material improve the properties of the composite material when combined in the composite.

In certain embodiments, the composite materials disclosed herein comprise composite materials that are in any form, e.g., in the form of strips, sheets, or tubes, and maintained in a lyophilized (e.g., freeze-dried) condition for storage. Before being used, the material may be rehydrated with sterile saline. When implanted in a subject, it is contemplated that the composite material is repopulated with host tissue by cellular regenerative repopulation assisted by enhanced blood vessel growth (i.e., revascularization) due to the geometry and positioning, i.e., as blood flows through the material, host tissue forms in the material. Thus, in certain embodiments, it is contemplated that the tissue becomes genetically identical to the host regardless of whether it is harvested from the human patient or made from another source.

### Method of Use

In certain embodiments, this disclosure relates to methods of repairing a tissue comprising implanting an effective amount of a composite material as disclosed herein into a subject at a location of desired tissue growth in a subject in need thereof and optionally suturing the composite material to the surrounding tissue or fascia. In certain embodiments, this disclosure relates to methods of repairing the heart following open-heart surgery such as intracardiac defects, septal defects and annulus repairs, cardiac and vascular reconstruction and repairs, peripheral vascular reconstruction and repairs, great vessel reconstruction and repairs, and suture-line buttressing.

In certain embodiments, the location of desired tissue growth is in the heart of a subject diagnosed with heart defect. In certain embodiments, the subject is less than one, two, or three years old or at risk of, exhibiting symptoms of, or diagnosed with congenital heart disease regardless of age.

In certain embodiments, a composite material disclosed herein is used in an atrial septal defect repair by implanting a patch of a composite material disclosed herein in, on, or around the atrial septum, i.e., the wall between the left and right atria (upper chambers) of the heart.

In certain embodiments, a composite material disclosed herein is used in a ventricular septal defect repair by implanting a patch of a composite material disclosed herein in, on, or around the ventricular septum.

In certain embodiments, a composite material disclosed herein is used in a tetralogy of Fallot repair by placing a patch in, on, or around the right ventricle and main pulmonary artery to improve blood flow to the lungs.

In certain embodiments, a composite material disclosed herein is used in coarctation of the aorta repair by cutting the narrow section of the aorta and widening it by inserting/implanting a tube made of composite material disclosed herein. In certain embodiments, in certain embodiments, a composite material disclosed herein is used in coarctation of the aorta repair by implanting a stent comprising a composite material disclosed herein into the narrow section of the aorta.

In certain embodiments, this disclosure relates to methods of repairing, treating or preventing cardiovascular disease or condition using composite materials disclosed herein comprising implanting a heart valve, artificial heart valve, comprising or coated with a composite material disclosed herein.

In certain embodiments, this disclosure relates to methods of surgically implanting a heart valve comprising composite material disclosed herein. In certain embodiments, the heart valve leaflets comprise a composite material disclosed herein.

In certain embodiments, this disclosure relates to methods of surgically implanting a transcatheter heart valve (e.g., transcatheter aortic valve) comprising composite material disclosed herein. In certain embodiments, the transcatheter aortic valve (TAV) leaflets comprise a composite material disclosed herein.

In certain embodiments, this disclosure relates to methods of regenerating an atrioventricular (AV) valve to replace a defective AV valve within a heart of a subject comprising removing a defective AV valve from the heart of the subject and implanting a composite material disclosed herein within the heart of the subject to regenerate a functional AV valve.

In certain embodiments, this disclosure relates to methods of treating or preventing cardiovascular disease or condition using composite materials disclosed herein comprising implanting a vascular patch comprising or coated with a composite material disclosed herein for uses in vascular reconstruction and repairs, peripheral vascular reconstruction and repairs, and suture-line buttressing.

In certain embodiments, composite materials disclosed herein may be used in/as wound dressings, dura repair, fistula plugs, myocardial patches, myocardial injections, heart valve repair, tympanoplasty grafts, nasal septal defect repair, hernia or body wall repair, hemostasis grafts, urology slings, tracheal grafts, esophageal grafts, lung patches, small bowel grafts, staple bolsters, nerve grafts, spinal cord repair, nerve cuff, nerve guide, pelvic floor grafts, amniotic sac patches, cornea repair, cartilage repair, bone repair, tendon/ligament repair, muscle repair, plastic and reconstructive surgery applications, lip augmentation, facial augmentation, nipple reconstruction, bile duct repair, ureter repair, urethra repair, and vascular access graft.

In certain embodiments, this disclosure relates to methods of improving hernia repair or hernia reinforcement using composite materials disclosed herein. In certain embodiments, methods treating or preventing a hernia comprising implanting a composite material disclosed herein to fill a gap between fascial edges of the abdominal wall. In certain embodiments, the composite material is laid over the peritoneal sac and sutured to surrounding tissue or fascia.

Performing bariatric stomach reductions typically use staples to shut a portion of the stomach. The staple line sometimes bleeds or leaks. In certain embodiments, it is contemplated that composite materials disclosed herein can be used as a reinforcing material to strengthen the staple line prior to closure. In certain embodiments, this disclosure relates to uses of composite materials disclosed herein in bariatric surgery wherein methods comprise contacting a composite material in an area of the stomach and suturing to the surrounding tissue or fascia.

In certain embodiments, it is contemplated that the composite material disclosed herein is fully resorbed after 12, 18, or 24 months. In certain embodiments, it is contemplated that the composite material disclosed herein prevents or reduces chronic inflammation, tissue deposition, and/or fibrosis, e.g., a fibrotic valve. In certain embodiments, it is contemplated that the composite material disclosed herein prevents or reduces calcification. In certain embodiments, it is contemplated that the composite material disclosed herein has a certain thickness to achieve adequate mechanical strength at the time of implantation and transitions to complete resorption of the material within 12, 18, or 24 months.

In certain embodiments, this disclosure relates to a composite material used for the repair or replacement of living tissue comprising a composite material disclosed herein.

In certain embodiments, this disclosure relates to a composite material colonized by the cells of the subject, providing a matrix or scaffold for growth of the cells.

In certain embodiments, this disclosure relates to a composite material wherein living cells are attach to or become attached to the composite material. For example, cardiac stem/stromal cells, stem cell antigen-1+(Scal+) and lslet-1+(Isl-1+) cells, mesenchymal stem cells, bone marrow-derived stem/progenitor cells, human fetal skeletal progenitor cells or human articular chondrocytes, or induced pluripotent stem cells. In certain embodiments, this disclosure relates to an implant comprising a composite material disclosed herein and secreted exosomes of bone marrow mesenchymal stem cells (BMSCS) or cardiac stem cells (CSCs).

In certain embodiments, this disclosure relates to a composite material incubated with suitable cells, in vitro, prior to use, to provide a composite material comprising tissue, which may be natural tissue or modified or genetically engineered tissue.

In certain embodiments, this disclosure relates to a composite material comprising DNA, RNA, proteins, peptides, or therapeutic agents for uses reported herein. In certain embodiments, the RNA is mircroRNA (miR), miR-24, miR-199a, or miR-590. In certain embodiments, protein is a growth factor, collagen, proteoglycan, glycosaminoglycan (GAG) chain, glycoprotein, cytokine, cell-surface associated protein, cell adhesion molecule (CAM), angiogenic growth factor, endothelial ligand, matrix metalloprotease, cadherin, immunoglobin, fibril collagen, non-fibrillar collagen, basement membrane collagen, small-leucine rich proteoglycan, decorin, fibromodulin, keratocan, lumican, epiphycan, heparan sulfate proteoglycan, perlecan, agrin, testican, syndecan, glypican, serglycin, selectin, lectican, aggrecan, versican, neurocan, brevican, cytoplasmic domain-44 (CD44), macrophage stimulating factor, amyloid precursor protein, heparin, chondroitin sulfate B (dermatan sulfate), chondroitin sulfate A, heparan sulfate, hyaluronic acid, fibronectin (Fn), tenascin, elastin, fibrillin, laminin, nidogen/entactin, fibulin I, fibulin II, integrin, a transmembrane molecule, platelet derived growth factor (PDGF), epidermal growth factor (EGF), transforming growth factor alpha (TGF-alpha), transforming growth factor beta (TGF-beta), fibroblast growth factor-2 (FGF-2) (also called basic fibroblast growth factor (bFGF)), thrombospondin, osteopontin, angiotensin converting enzyme (ACE), and vascular epithelial growth factor (VEGF).

In certain embodiments, this disclosure relates to a composite material used for creating a stent comprising the composite material disclosed herein.

In certain embodiments, this disclosure relates to a composite material disclosed herein attached to permanent support such as a steel plate or pin wherein the structural support remains while the composite material acts as a scaffold and integrates following growth of tissue.

In certain embodiments, this disclosure relates to a composite material disclosed herein placed within a subject when a tissue or an internal tissue or organ is being repaired. In certain embodiments, this disclosure relates to a composite material disclosed herein placed on the body of a subject when a cut, wound, or skin tissue is being repaired.

In certain embodiments, this disclosure relates to methods of implanting a composite material disclosed herein in a subject and administering an anticoagulant or immune suppressive agent to the subject.

### Cell Honing Cardiovascular Tissue Substitute

One object of this disclosure is to optimize the safety and functional efficacy of hybrid composite materials disclosed herein for cardiovascular applications. Cardiovascular tissue substitutes which switch from an inert implant to one that integrates into the host and remodels, are needed, especially for children undergoing cardiac surgery for the repair of congenital heart defects. Biohybrid materials containing a native matrix core, and engulfed in a cell-honing, degradable, polymer, were used to achieve host integration of the implanted material. In one example, decellularized bovine pericardial extracellular matrix was used as the core, with 70 μm thick matrix of polycaprolactone-chitosan electrospun onto it.

Spectroscopy of the material cross-section depicted new amide chemical bond formation and C—O—C stretch between the matrix core and polymer overlay. Mechanical strength and extensibility ratio of the matrix alone was 18,000 plus/minus 4,200 KPa and 0.18 plus/minus 0.03% and the composite was 20,000 plus/minus 6,600 KPa, 0.35 plus/minus 0.20% indicating an increase in strength and extensibility.

In rats implanted with the material subcutaneously, cellular infiltration into the core was significantly higher in the biohybrid, compared to the core alone or other alternatives. The material was then implanted in 3 juvenile sheep as a left atrial patch, a carotid patch, and a pulmonary artery patch, and the animals were followed to 3 months. At explant, all implants were thrombus free, depicted cellular infiltration with macrophage honing, and remodeling with the matrix proteins. This biohybrid material has the potential to hone host cells and remodel without structural deterioration indicating its potential use as an active cardiovascular tissue substitute. This active cardiovascular tissue substitute contains a matrix core having a physiological architecture with a polymer covering to hone host cells into the matrix. Such a host integrating material can yield a better material substitute for pediatric cardiac surgery.

Characterization of the composite material depicted new chemical bonds between the matrix and polymer layers, without delamination, and adequate mechanical strength. In an established subcutaneous rat model, the unique material honed cells significantly better than the alternatives, with the density and distribution of cells within the material being superior.

In a chronic juvenile sheep study, the material was non-thrombogenic and had adequate mechanical strength to sustain both arterial and venous hemodynamics. Explants from the sheep did not show calcification, and depicted regenerative M2 macrophages, and new matrix protein deposition were indicative of remodeling. This material can be used as an active cardiovascular tissue substitute for potential clinical applications.

Active cardiovascular tissue materials, which are inert at the time of implantation, but over time hone the cells of the host to remodel into tissue of the patient, e.g., fully replaced, have been contemplated for cardiovascular surgery, especially in children undergoing cardiac surgery. However, synthetic materials typically lack durability or thrombose over time. Thus, the need for improved active cardiovascular tissue materials is immense. Autogenous tissue formation from implanting synthetic mandrels covered with Dacron™ into subcutaneous pockets forms on the mandrels over 5-10 weeks. The resulting grafts lacked controllable thickness, have poor hemostasis at some regions or aneurysmal degeneration. Creating heart valves in a subcutaneous pocket may be accomplished by using mandrels with surface modifications that enhanced tissue deposition. However previous attempts lacked compliance and were often too stiff for use as heart valve leaflets. See Hayashida et al. J Thorac Cardiovasc Surg 2007, 134(1):152-9.

Electrospinning of various supramolecular polymers enables the development of programmable materials for host cell honing. Such materials can utilize a three phase in-vivo material remodeling process: (1) implantation of the material into the body of a patient; (2) neo-tissue formation onto the material; and (3) functional restoration. Host tissue formation onto the implant may be observed with collagen deposition at the site of the implant matrix resorption.

Experiments were performed to determine whether endogenous tissue formation may be better achieved, if the resorbable, cell honing material is very thin, and the attracted cells hone into an environment with native architecture. Thus, in certain embodiments, a biohybrid material was developed containing a decellularized extracellular matrix core that has native 3-dimensional architecture and is overlaid with cell-honing polymeric nanofibers of blended polycaprolactone and chitosan. The relative thickness of the matrix core is significantly higher than the polymeric layer, thus reducing the time for the polymer to degrade. The cells infiltrating into the native architecture matrix may have a higher probability to function as in their native environment, and potentially reduce the risk of calcification and fibrosis. Decellularized matrix core was chosen to mimic the native architecture, whereas the blend of polycaprolactone and chitosan was chosen to provide a hydrophilic layer that is biocompatible, hemocompatible and slow degrading that can potentially match the in-vivo deposition of neo-extracellular matrix.

The biohybrid material and its mechanical strength were characterized using a variety of testing methods: hemocompatibility in blood flow loops with high and low shear stresses and flow disturbances, biocompatibility and cell honing potential in a subcutaneous rat model, and 3-month long safety and feasibility in juvenile sheep implanted with the material in both low-pressure venous, and high-pressure arterial environments.

### Matrix-Polymer Composite Material (Bio-Hybrid)

Bovine pericardium (BP) was sourced from a commercial vendor and the Bio-Hybrid material was prepared. BP was decellularized with 2% sodium deoxycholate (average MW 1200-5000) for 48 hours, followed by 1% sodium deoxycholate for 24 hours, and treatment with DNase and RNase for 2 hours at 37° C. in a shaker incubator. Acellularity was confirmed by DNA estimation, histology (hematoxylin and eosin (H&E) and 4,6-diamidino-2-phenylindole (DAPI) staining) and Scanning Electron Microscopy (SEM). Polycaprolactone 12% (mol wt. 70,000-90,000) and 1% chitosan (mol wt. 190000-375000 Da) blend was prepared in a mixture of 80:20 trifluoroacetic acid and dichloromethane. The polymer solution was then electrospun onto the decellularized pericardium core that was mounted on a rotating mandrel using a custom build setup. Polymer fibers were deposited in the circumferential direction of the decellularized pericardium for 3 hours, at room temperature. The sample was then neutralized in 0.5M NaOH for minutes to enable the free amine of the chitosan to interact with the decellularized tissue. The sample was then washed in distilled water and preserved in 70% ethanol.

### Integrity of the Decellularized Core and Core-Polymer Interaction

Structural integrity of the decellularized matrix core was assessed by staining for collagen, elastin and GAGs using trichrome, Verhoeff's van Gieson, and Alcian blue stains. Quantitative estimation of collagen, elastin and GAGs was performed with hydroxyproline assay, Fastin™ Elastin assay, and dimethylmethylene blue (DMMB) assay respectively. Polymer-core interface was visually assessed with SEM, and molecular interactions were quantified with Fourier Transform Infrared spectroscopy (FT-IR) and X-ray photon spectroscopy (XPS). FT-IR spectra was recorded for PCL, Ch, PCL-Ch blend, decellularized BP and Bio-Hybrid samples to identify the differences in their functional groups. XPS was used to determine the elemental and chemical composition of each material.

### Core-Polymer Peel and Shear Strength

Strength of the polymer-core interaction was quantified using two methods—a setup to measure the tangential peel force required to delaminate the polymer off the decellularized core, and a second experiment in which tubes of the material were prepared and mounted into a dynamic flow loop to induce shear stress on the polymer. The peel force was plotted against time, and the instance of peeling was defined as a point when a sharp change in the force-displacement curve was observed. Peel strength was then calculated as the load imposed tangentially at the time of peel, to the longitudinal cross-sectional area of the material (width×length). In the shear stress experiments, a shear stress of 15 dynes/sq cm was imposed on the inner walls of the tube made from this material, with a glycerin-water mixture that was equivalent to blood viscosity. The conduits remained in the flow loop with steady flow for 24 hours, after which they were extracted and examined with SEM. In a parallel experiment, the conduit was constricted to form a 50% stenosis, to create a high shear jet, and recirculating zones, and the experiment was repeated.

### Yield Properties of the Bio-Hybrid

Uniaxial testing was performed on the Bio-Hybrid material (n=7) and compared to untreated BP (n=7) and decellularized BP (n=7). For uniaxial testing, a dog bone shape of the material was cut (5 mm×30 mm), with the direction of the fibers aligned along the sample. The thickness of the sample was measured with a screw gauge and averaged over multiple locations, and graphite markers were placed on the sample. In a universal testing machine, the sample was first preconditioned for 10 cycles and then pulled to failure at a strain rate of 10 mm/min. The strain and load data were measured, and the stress-strain curves were plotted. From the stress-strain curves, the uniaxial ultimate tensile strength, ultimate tensile extensibility, upper and lower tangent modulus were calculated and compared between the groups.

### Biaxial Mechanical Characterization

Biaxial mechanical testing was performed on the three groups of tissues, to quantify their mechanical properties. Bio-Hybrid material (n=13), untreated BP (n=13), and decellularized BP (n=13) were evaluated in a biaxial mechanical tester. Samples (6 mm×6 mm) were cut, the tissue thickness was measured with a screw gauge at five different regions, and the sample was mounted onto the biaxial tester using BioRakes™ positioned along each edge of the tissue. The samples were immersed in phosphate buffered saline maintained at 37 degrees C. Equibiaxial testing was performed by stretching the samples to 10% strain uniformly in both directions, and step biaxial testing was performed at 10% and 20% strain by stretching in one direction and holding the other stationary. In both protocols, tissues were preconditioned for the first 7 cycles and then data was acquired. Marker displacement was used to calculate Green strain and 2nd Piola Kirchhoff stress along the tested directions, and the stress-strain curves were plotted.

### Biocompatibility Studies

Biocompatibility of the material was investigated using in-vitro cell culture and in-vivo rat subcutaneous implants. For the in-vitro cell culture studies, porcine mitral valve endothelial and interstitial cells were isolated and seeded onto the material. Forty-eight hours after seeding, the materials were fixed and stained with rhodamine phalloidin, and DAPI. Retainment and viability of cells was observed under a microscope. In-vivo biocompatibility was assessed by implanting Bio-Hybrid material (n=16), Glut fixed untreated BP (n=15), and decellularized BP (n=15) in sub-cutaneous pockets. Sprague Dawley rats (200-300 g) were purchased (50% male and 50% female), and the implantation was performed under anesthesia with 2% isoflurane mixed in 100% oxygen, using aseptic techniques. With the animal maintained on anesthesia with a nose cone, 1 cm subcutaneous pockets were made on the dorsal side of the rat. Each rat received one of the materials, and 10 samples of that chosen material were implanted, such that each subcutaneous pocket was separated from one another. Incisions were closed with 4-0 Vicryl™ suture and the animals were recovered with appropriate pain medications. After surgery, five rats were terminated in each group at 1 week (n=5), 4 weeks (n=5), and 12 weeks (n=5) to evaluate the material. At termination, a midline incision was made in the dorsal region to carefully expose all the implanted materials, a steel hemostatic clip was placed at an edge to identify the skin facing side from the muscle facing side, and the tissues were then stored in 10% buffered formalin. Histology was performed by staining with hematoxylin and eosin, Movat's Pentachrome and von Kossa, to assess cellular infiltration, ECM remodeling and calcification, respectively.

### Hemocompatibility

Hemocompatibility of the Bio-Hybrid and decellularized BP were assessed by percentage hemolysis assessment assay, clot formation assay, and platelet adhesion assay. Fresh porcine whole blood was collected with EDTA (1.6 g/L) and maintained under constant agitation. For percentage hemolysis studies, sterile samples were incubated with 5 ml whole blood for 30 minutes at 37° C. 1 cc of blood was sampled at baseline, and then at the end of the experiment, and percentage hemolysis was calculated as (Free Hb/Total Hb)*100. To assess clot formation, the decellularized BP and the Bio-Hybrid samples were incubated in constantly agitated whole blood at 37 degrees C. for 30 min. Clot formation was assessed visually. For platelet adhesion assay, platelets were isolated from 30 ml whole blood by centrifugation at 2000 rpm for 12 min, and the supernatant centrifuged at 5000 rpm for 15 min. The platelet pellet was re-suspended in 2 ml of platelet poor plasma and 500 μL of platelet suspension was added to the samples. Samples were kept in shaker incubator for 30 min at 37° C. at 100 rpm, fixed in formalin for 30 min and stored in 70% ethanol. SEM was used to image adhered platelets on the surface of these materials.

### In-Vivo Juvenile Sheep Studies

Three 25 kg Suffolk male sheep were implanted with the Bio-Hybrid material as patches in their carotid artery, left atrium, and main pulmonary artery that mimic different flow conditions. Patches were implanted into each location using a side-biting clamp, incising into the native tissue, and suturing a patch into the incision. Blood was drawn at the beginning of the procedure, at closure, 1 day after the procedure, at weekly intervals for the first month, and at monthly intervals until termination. The animals were euthanized with 80-150 mEq Euthasol™, and the left carotid artery, main pulmonary artery and the left atrial patches were carefully dissected along with surrounding native tissue. The explants were analyzed with histology using H&E, Movat's Pentachrome and von Kossa stains for cellular infiltration, immune response, presence of endothelium, matrix remodeling and calcification of the material. Blood was analyzed for total red blood cell and white blood cell (WBC) counts, differential count, platelets, D-dimer, and lactate dehydrogenase (LDH). Immunohistochemistry was done on the explants to identify the type of inflammatory macrophages phenotype (M1-iNOS and M2-CD163 macrophages), presence of a smooth muscle actin cells (α-SMA) and cellular damage using vimentin.

### Polymer-Tissue Interface Characterization

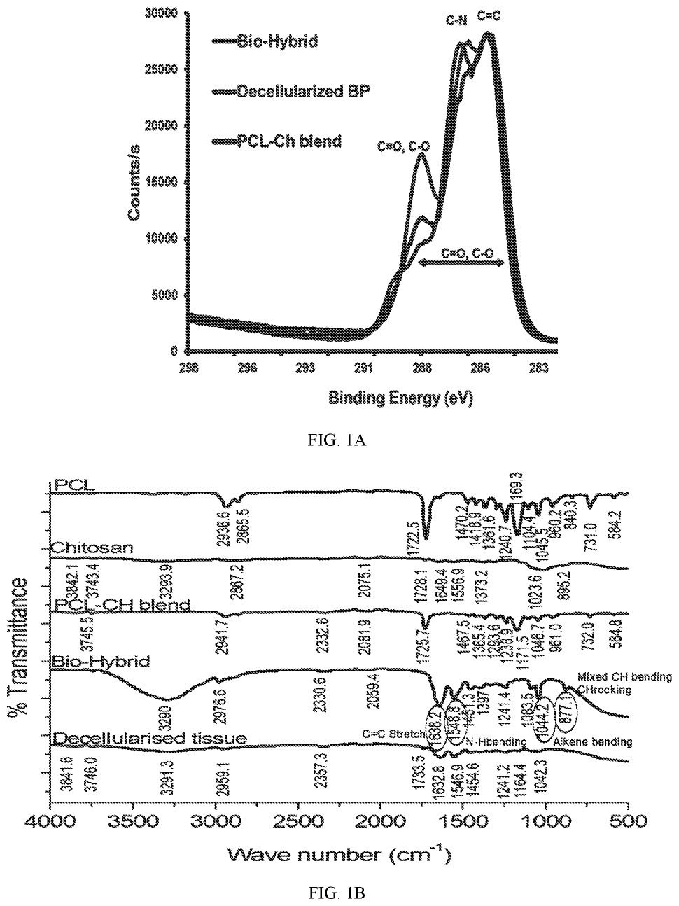

SEM images of the untreated BP, decellularized BP, and the Bio-Hybrid were obtained. Bovine pericardial surface depicts cells integrated with the fibers, whereas decellularization removed the cells while preserving the matrix architecture. The Bio-Hybrid surface depicts nanofibers overlaid on the core and covering. Images show the interface between the polymer and the matrix core, and a SEM image of the cross section depicting adherence of the polymer to the underlying matrix core. XPS results of each of the materials is shown in FIG. 1A. In the Bio-Hybrid material, new peaks corresponding to C═O, C—O, and C—N were observed, which were not seen in the decellularized BP alone or the polymer blend alone. FT-IR data in FIG. 1B depicts the spectra of the newly formed chemical bonds between the polymer and the underlying ECM. Two new peaks at 1548.8 cm−1 and 1638.2 cm−1 corresponding to amide groups, and at 877.1 cm−1 to 1044.2 cm−1 corresponding to C—O—C stretch were measured.

### Peel Strength of the Bio-Hybrid

Results of the peel strength and shear induced delamination experiments are shown in FIG. 2. The load required to peel the polymer from the surface of the matrix core is depicted in from four distinct Bio-Hybrid samples, with the force required to peel ranging from 40-75 grams, with an average peel strength of 56.25 grams. At 15 and 30 dynes/cm 2 of shear stress, disarray of the decellularized fibers was observed, with higher damage associated with higher shear rates. In the Bio-Hybrid, the polymer nanofibers did not peel or disrupt, but formed a more uniform layer on the tissue, aligned along the flow direction.

### Mechanical Testing of the Bio-Hybrid Composite Material

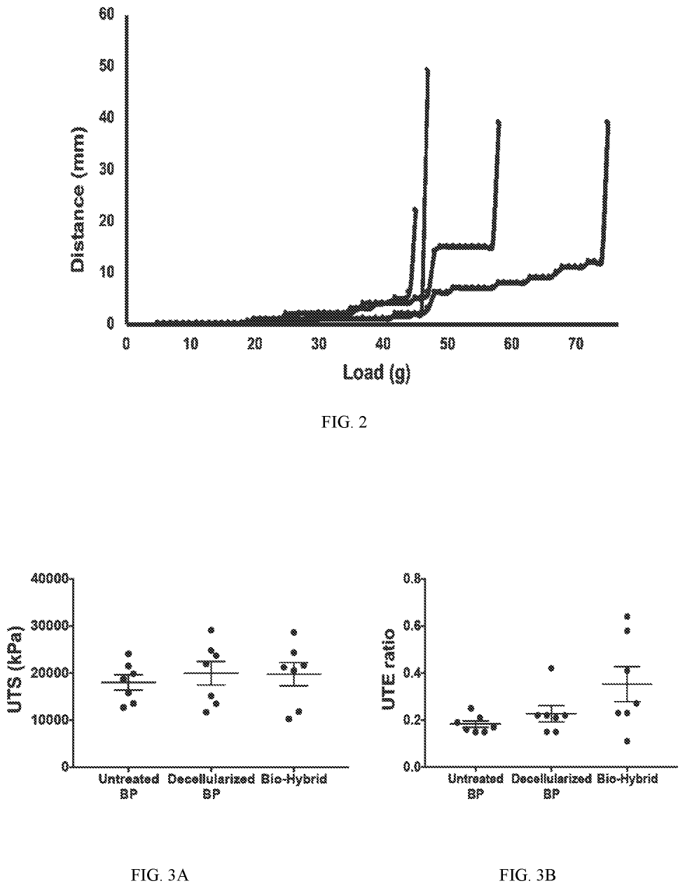

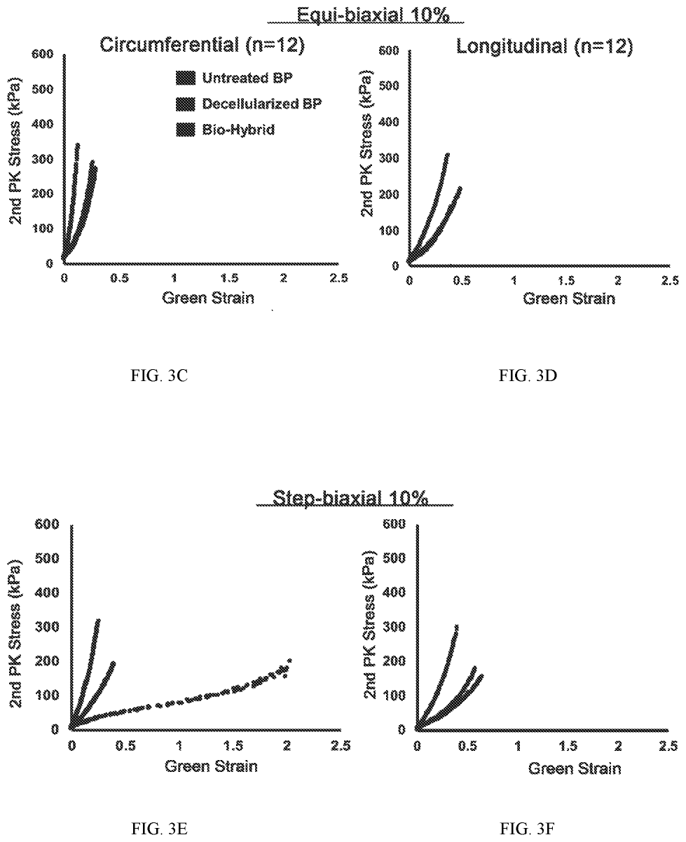

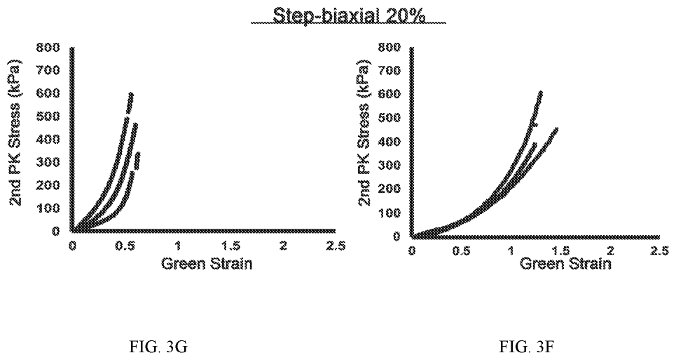

The uniaxial mechanical testing of the untreated BP, decellularized BP, and Bio-Hybrid samples did not show any difference in the ultimate tensile strength, as shown in FIG. 3A (untreated BP: 18000 plus/minus 4200 kPa, decellularized BP: 20000 plus/minus 6600 kPa and Bio-Hybrid: 20000 plus/minus 6600 kPa). However, there was a significant increase in the tensile extensibility (FIG. 3B) of the Bio-Hybrid compared to the untreated BP (untreated BP: 18 plus/minus 3.7%, decellularized BP: 23 plus/minus 9% and Bio-Hybrid: 35 plus/minus 2%). Results from equibiaxial testing in the circumferential and longitudinal directions are depicted in FIG. 3C-D, wherein the Bio-Hybrid material was stiffer than the decellularized and untreated BP in both directions. Stiffness did not differ between the decellularized and untreated BP, indicating preservation of the ECM after the detergent wash. In step biaxial testing at 10% stretch, Bio-Hybrid material remained the stiffest among the tested materials (FIG. 3E-F). At 20% stretch, the Bio-Hybrid was stiffest only in the circumferential direction, but not in the longitudinal direction (FIG. 3G-H).

Biocompatibility, inflammation, and calcification of Bio-Hybrid

The in-vitro biocompatibility studies of the Bio-Hybrid composite indicate the Bio-Hybrid and decellularized BP showed similar and better attachment of porcine valve endothelial and interstitial cells, whereas the Glut fixed untreated BP (Glut-BP) did not show any attachment of cells evident by the absence of fluorescent signal on these samples. The temporal changes were evaluated in the histopathology of Glut-BP, decellularized BP and the Bio-Hybrid at 1, 4 and 12 weeks upon implantation in subcutaneous pockets in rats. H&E staining of the samples at 12 weeks depicts significant cellularity across the cross section of the Bio-Hybrid material. Cellularity was observed in the decellularized BP as well, but it was sparse. In the Glut-BP, cellularity was confined to the surface where the sample interfaced with the subcutaneous tissue. The longitudinal changes that occurred in cellularity from H&E between the three materials in this rodent model demonstrated the extent of cellularity among different groups. Longitudinal differences in the ECM between materials were evaluated with pentachrome staining at 1-, 4- and 12-week timepoints. In the Glut-BP, yellow staining is predominant, which depicts collagen. In the decellularized BP, green staining is prominent, which indicates mucin deposition. In the Bio-Hybrid material, a combination of yellow, green, and black staining is evident, indicating presence of collagen, mucin, and elastin. Staining (von Kossa) depicts the longitudinal changes in calcification at the same timepoints that depicted black spots on the surface of the Glut-BP, but not in the other materials. The presence of the polymer was seen in the explants at 12 weeks in the Bio-Hybrid samples demonstrating slow degradation profile.

### Hemocompatibility of Bio-Hybrid:

Table 1 below show data on the in-vitro hemocompatibility of the Bio-hybrid using three different tests. The Bio-Hybrid and the decellularized BP samples did not show any hemolysis (0 g/dL) of cells upon agitating with fresh blood. There was also no clot formation on the decellularized BP and Bio-Hybrid samples demonstrating unchanged hemocompatibility of the Bio-Hybrid with the addition of polymer to the decellularized core. Additionally, there was minimal platelet adhesion on the Bio-Hybrid in comparison to the decellularized BP core in the scanning electron microscopic images.

TABLE 1
shows the percentage hemolysis (free
Hb/total Hb)*100—after 30 min of incubation

| | Total Hb | Free Hb | % |
|---|---|---|---|
| Sample | (g/dL) | (g/dL) | Hemolysis |
| Decellularized | 14.7 ± 0.1 | 0 | 0 |
| Bio-Hybrid | 15.26 ± 0.15 | 0 | 0 |

### In-Vivo Juvenile Sheep Cardiovascular Implant Studies

Bio-Hybrid patches were explanted at 90 days (12 weeks) after implantation in a juvenile sheep in the carotid artery, main pulmonary artery, and left atrium. Three sheep were implanted in total, and all animals survived the 90-week duration without any adverse events. At explant, none of the tissues dehisced, tore, or demonstrated any thrombus formation. Under direct vision, the blood facing side of the Bio-Hybrid was glistening with a thin layer of tissue formation. Flow at the patch site assessed using color Doppler and angiography confirmed the hemocompatibility of the Bio-Hybrid material without any clot or thrombus formation on all the implants. Histopathological analysis was performed of the explants from carotid, pulmonary artery, and left atrial regions. H&E staining depicts cellular infiltration into the Bio-Hybrid material, across its entire thickness and was comparable to the native tissue from the surrounding region. Pentachrome staining depicted mucin deposition, with homogenous green staining of the sample. Staining (von Kossa) did not demonstrate any calcification in any of the explanted samples. The histology after 90 days for the other two animals demonstrated cellular infiltration and matrix remodeling without any calcification in the material.

Immunohistochemistry of the explants was performed from the three regions. Vimentin positive cells were observed in tissues explanted from the three distinct regions, indicating their viability and lack of any toxic effects of the material on their survival. Among the infiltrating cells, α-smooth muscle actin positive staining was abundant in the carotid and pulmonary artery explants, but not in the left atrial explant. iNOS positive cells, which are markers of pro-fibrotic M1 macrophages, were high in both the carotid and pulmonary artery explants. However, in parallel M2 macrophages (CD163) were also abundant in these tissues, indicating a potential shift from inflammatory to a more reparative phenotype at this time point.

Temporal changes in the systemic inflammatory response observed in total and differential blood count, platelets, D-dimer and LDH for all the three sheep were evaluated. The total WBC count increased to 8.83±2.69×10³/μL (normal range: 4-8×10³/μL) by day 7, which came down to normal range by 2 months. The average percentage of neutrophils also increased from 45.00±6.42% to 59.67±6.11% immediately after surgery but normalized after day 1 and remained normal throughout the 12 weeks. The average lymphocyte count remained normal until day 21, after which it remained slightly elevated for the rest of time points until termination. Other parameters that were measured from blood draws were platelets, D-dimer, and LDH that are indications of ongoing thrombocytosis and cellular damage. An increase in the above parameters for a brief period after surgery is normal. The platelet counts increased slightly compared to baseline after surgery (1074.33±340.99×10³/ul) but was within normal limits (800-1100×10³/ul) for all the three sheep. Average D-dimer values remained high during follow up since one of the sheep had a high baseline D-dimer value (946 ng/mL). In this animal, a thrombus was dislodged to the carotids when first patched in this animal. LDH increased after surgery and normalized after day 21.

What is claimed is:

1. A composite material comprising a first material and a second material coated on the first material,
wherein the first material is an inert substantially non-biodegradable material providing mechanical support;
wherein the second material is an active biodegradable material attracting cells from the body of a subject; and
wherein when the composite material is present in tissue of the subject the inert material attracts cells from the active material providing cells which hone into the inert material providing a remodeled material.

2. The composite material of claim 1, wherein the composition and proportion of the inert and active material determines the remodeling of the material in a living system.

3. The composite material of claim 1, wherein the inert material is a synthetic or natural polymer.

4. The composite material of claim 1, wherein the inert material is derived from tissues of an animal or human.

5. The composite material of claim 1, wherein the active material is a synthetic or natural polymer.

6. The composite material of claim 1, wherein the active material elutes a drug.

7. The composite material of claim 1, wherein the active material is hemocompatible.

8. The composite material of claim 1 made by the process of dipping inert material into the active material.

9. The composite material of claim 1 made by the process of overlaying the active material is on the inert material.

10. The composite material of claim 1 made by the process of weaving strands of the inert and active material with one another.

11. The composite material of claim 1, wherein the inert material is decellularized pericardium from an animal or a human.

12. The composite material of claim 11, wherein the active material is a polymer fiber of caprolactone and chitosan

13. The composite material of claim 11, wherein the polymer fiber of caprolactone and chitosan fiber is about a 10:1 by weight ratio.

14. The composite material of claim 12, wherein the active material has an average thickness of between 70 to 100 μm.

15. The composite material of claim 11 made by the process of contacting the decellularized pericardium with a dialdehyde providing aldehyde functionalized decellularized pericardium, and contacting the aldehyde functionalized decellularized pericardium with a polymer fiber of caprolactone and chitosan.

16. A method of repairing a tissue comprising implanting an effective amount of a composite material of claim 1 into a subject at a location of desired tissue growth in a subject in need thereof.

17. The method of claim 16, wherein the location of desired tissue growth is in the heart of a subject diagnosed with a heart defect.

18. The method of claim 16, wherein the subject is diagnosed with a congenital heart defect.
